(19) United States
(12) Patent Application Publication
(10) Pub. No.: US 2006/0292087 A1
(43) Pub. Date: Dec. 28, 2006

(54) Dentrifrice and method of making the same

(76) Inventor: James Moore, Tucson, AZ (US)

Correspondence Address: MICHAEL WINFIELD GOLTRY, 4000 N. CENTRAL AVENUE, SUITE 1220, PHOENIX, AZ, 85012, US

(21) Appl. No.: 11/168,281

(22) Filed: Jun. 28, 2005

(51) Int. Cl. A61K 8/21; A61K 8/30

(52) U.S. Cl. 424/49; 424/52; 424/53

(57) ABSTRACT

A dentifrice includes a carrier material and a plurality of particles in the carrier material. The plurality of particles includes multiple regions of different colored materials and an outer region which at least partially encapsulates the multiple regions of different colored materials. The multiple regions of different colored materials dissolve in response to a trigger. Each region dissolves in a predetermined amount of time so that the color of the dentrifrice indicates how long a person has been brushing his or her teeth.

### BACKGROUND OF THE INVENTION

1. Field of the Invention

This invention relates to dental hygiene and, more particularly, to a dentrifrice which reinforces recommended brushing techniques.

2. Related Art and Prior Art Statement

It is well known to apply a dentrifrice to ones teeth to aid in cleaning them. The cleaning is typically done by brushing the dentrifrice on the teeth using a tooth brush. This process is routinely done daily or more often in order to remove food particles, plaque, and other debris from around the teeth and gums, and to leave the mouth with a pleasant and refreshing feeling. Although there are many varieties of dentrifrice to choose from, the ones that are commercially available do not reinforce recommended brushing techniques.

For example, most dental hygiene professionals recommend brushing the teeth for a certain amount of time, which can be between two and three minutes, for example. This facilitates efficient cleaning of the teeth and gums. However, it is often difficult to keep track of the brushing time. This is particularly difficult for children, which shifts the onus to parents to make sure that their children are adequately brushing their teeth. As a result, there is a failure to minimize cavities and other problems associated with poor or unsupervised brushing habits. Consequently, there is a need for a dentrifrice that can reinforce recommended brushing techniques. In particular, there is a need for a dentrifrice that can indicate the time that the teeth have been brushed.

### BRIEF SUMMARY OF THE INVENTION

The present invention provides a dentifrice, which includes a carrier material and a plurality of particles in the carrier material. The plurality of particles includes multiple regions of different colored materials and an outer region which at least partially encapsulates the multiple regions of different colored materials.

The present invention also provides a method of cleaning teeth, which includes providing a tooth brush with a plurality of bristles; positioning a dentrifrice on the bristles of the tooth brush; and applying the dentrifrice to the teeth with the plurality of bristles, the particles in the plurality of particles reflecting light from the light source and changing color as they dissolve. In some embodiments, the tooth brush includes at least one light source which emits light through the plurality of bristles. The dentifrice includes a carrier material and a plurality of particles in the carrier material. The plurality of particles includes multiple regions of different colored materials.

The present invention further provides a method of making a dentrifrice which includes providing a carrier material; providing a plurality of particles in the carrier material, the plurality of particles including multiple regions of different colored materials; and mixing the plurality of particles and the carrier material together.

These and other features, aspects, and advantages of the present invention will become better understood with reference to the following drawings, description, and claims.

### BRIEF DESCRIPTION OF THE DRAWINGS

Referring to the drawings:

FIG. 1 is a simplified perspective view of one embodiment of a tooth brush and dentrifrice in accordance with the present invention;

FIG. 2a is a simplified partially cut-away view of an embodiment of a particle included in the dentrifrice of FIG. 1;

FIG. 2b is a simplified side view of the particle of FIG. 1;

FIGS. 3a through 3f are simplified perspective views of the particle of FIG. 2;

FIGS. 4a through 4c are simplified graphs showing how the particle changes color as a function of time;

FIG. 5 is a simplified graph showing how the thickness of a material region is related to the color change time;

FIG. 6a is a simplified perspective view of another embodiment of a particle in accordance with the present invention;

FIG. 6b is a simplified partial cut-away view of the particle of FIG. 6a;

FIGS. 7a and 7b are simplified side and perspective views, respectively, of one embodiment of the tooth brush of FIG. 1 in accordance with the present invention;

FIG. 7c is a simplified side view of another embodiment of the tooth brush of FIG. 1 in accordance with the present invention;

FIG. 8 is a simplified perspective view of another embodiment of a tooth brush in accordance with the present invention; and

FIG. 9 is a simplified perspective view of another embodiment of a tooth brush in accordance with the present invention.

### DETAILED DESCRIPTION OF THE INVENTION

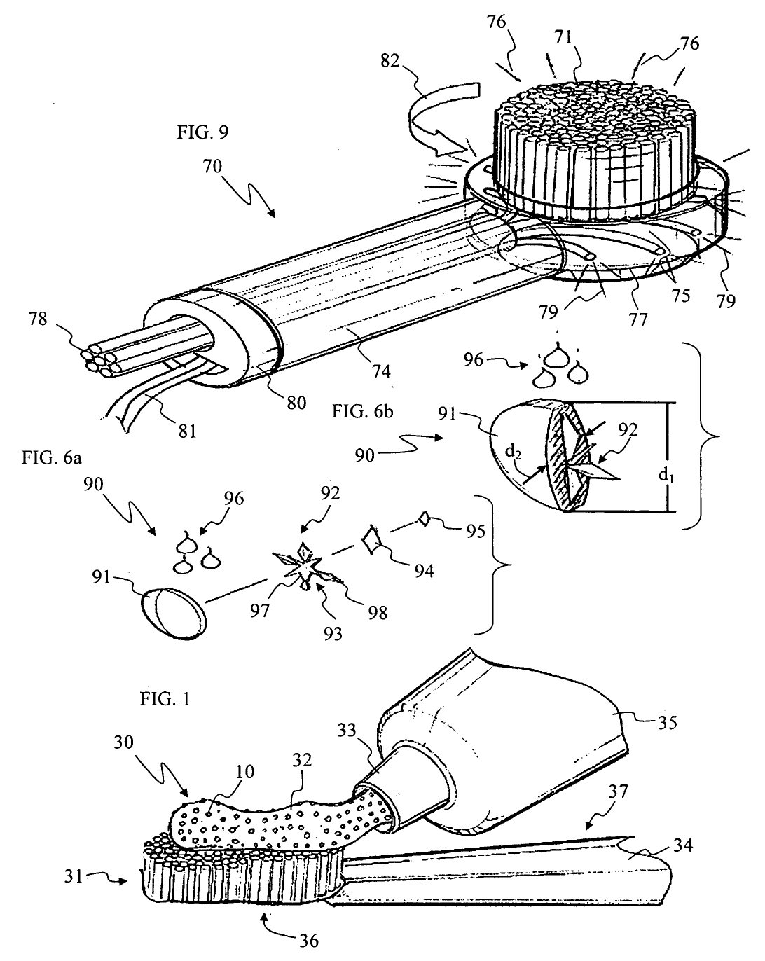

FIG. 1 is a simplified perspective view of one embodiment of a tooth brush 37 and a dentrifrice 30 in accordance with the present invention. It should be noted that like reference characters indicate corresponding elements throughout the several views. In this embodiment, tooth brush 37 includes a neck portion 34 which carries bristles 31 near an end 36. Dentrifrice 30 is typically ejected from a tube 35 through a nozzle 33 onto bristles 31. In accordance with the invention, dentrifrice 30 includes a carrier material 32 with particles 10 mixed therein. Carrier material 32 can include tooth paste or another material used to clean teeth and particles 10 can include an edible material, such as gelatin. In a typical use, dentrifrice 30 is applied to the person's teeth with bristles 31 and then they are cleaned by moving the bristles across the teeth in a brushing motion. As will be discussed in more detail below, dentrifrice 30 reinforces recommended brushing techniques because particles 10 change color as a function of time as the teeth are brushed. Hence, the person brushing his or her teeth can tell the amount of time that has elapsed by the color of particles 10.

Particles 10 change color because they include multiple regions of different colored materials that dissolve with time in response to a trigger. In this way, particles 10 change color as a function of time in response to the trigger. The trigger can include moisture or friction, for example, or another mechanism which particles 10 changes color in response thereto. The change in response to moisture can be from the moisture dissolving particle 10. However, the response can also be from other mechanisms, such as a change in the pH of the moisture. The change in response to friction can be from bristles 30 mechanically forcing particles 10 against the person's teeth, which can be adjusted by the brushing motion used.

In one example, particles 10 include a blue core material region encapsulated by a yellow material region. A red material region is then positioned around the yellow material region. Hence, when the person starts brushing his or her teeth, particles 10 turn red, then yellow, then blue as the teeth are brushed and the red, yellow, and blue material regions dissolve. In one example, particles 10 can be red for about 45 seconds, yellow for about 45 seconds, and blue for about 45 seconds, so that the total brushing time is about 2 minutes and 15 seconds. In some examples, an outer encapsulation region can be positioned around the red material region. The outer encapsulation region can include a material which protects the other regions from undesirably losing their color or otherwise deteriorating before use. This is particularly useful during storage, such as before tube 35 has been purchased or before dentrifrice 30 has been used. The outer encapsulation region can be the same color as carrier material 32 or it can be a different color.

In accordance with the invention, the thickness of the red, yellow, and blue material regions can be chosen to provide a desired time. For example, if the red material region is made thicker, then particles 10 will be red for a longer period of time. If the red material region is made thinner, then particles 10 will be red for a shorter period of time. It should be noted that the same is true for the blue and yellow material regions. In this way, the total brushing time can be adjusted.

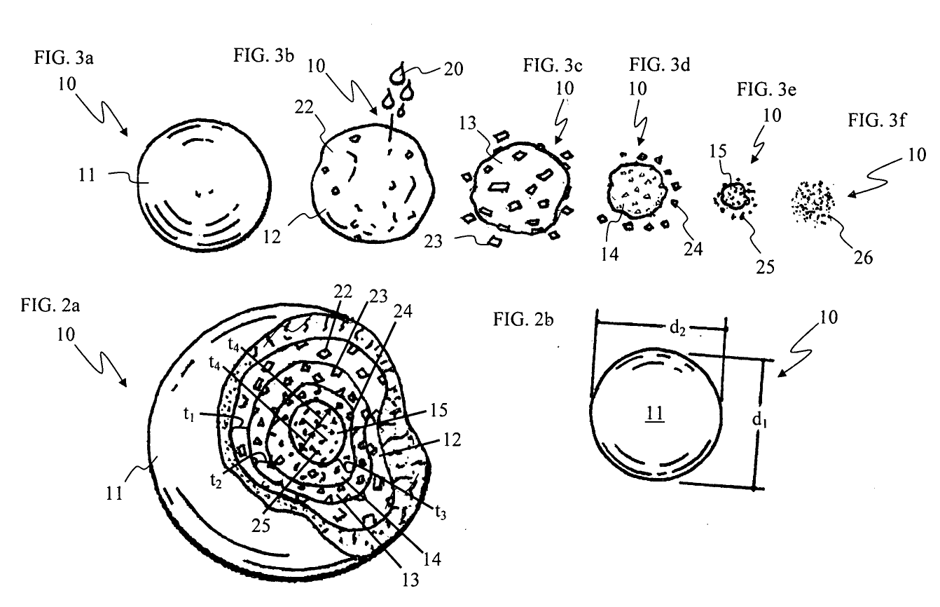

FIG. 2a is a simplified partially cut-away view of an embodiment of particle 10 included in dentrifrice 30 of FIG. 1 and FIG. 2b is a simplified side view of particle 10. In this particular embodiment, particle 10 includes a blue core material region 15 which is encapsulated by a green material region 14. A yellow material region 13 is positioned around green material region 14 and a red material region 12 is positioned around yellow material region 13. An outer encapsulation region 11 is positioned around red material region 12. In this particular example, regions 11-15 are spheroid in shape, with the shapes being substantially concentric relative to a center of blue core material region 15. FIG. 2b shows that particle 10 has a diameter d₁ and a diameter d₂. Diameters d₁ and d₂ can be the same so that particle 10 is spherical, but in other examples, diameters d₁ and d₂ can be different so that particle 10 has another shape, such as ellipsoidal. In still other examples, particle 10 can be square, rectangular, triangular, etc.

Material regions 12-15 include different colored materials, which can be colored in many different ways. In this example, they are colored by including light reflecting materials, such as edible glitter. For example, material regions 12, 13, 14, and 15 include light reflecting materials 22, 23, 24, and 25, respectively, wherein materials 22-25 each reflect a different color of light. Here, materials 22, 23, 24, and 25 reflect red, yellow, green, and blue light, respectively. In other examples, material regions 12-15 can include colored gelatin. Material regions 12, 13, 14, and 15 have thicknesses t₁, t₂, t₃, and t₄, respectively. Thickness t₄ of material region 15 corresponds to a radius of material region 15, although it can be another measure of region 15 in other examples. In this particular example, thicknesses t₁, t₂, t₃, and t₄ are not uniform around the periphery of their corresponding material regions, but they can be in other examples.

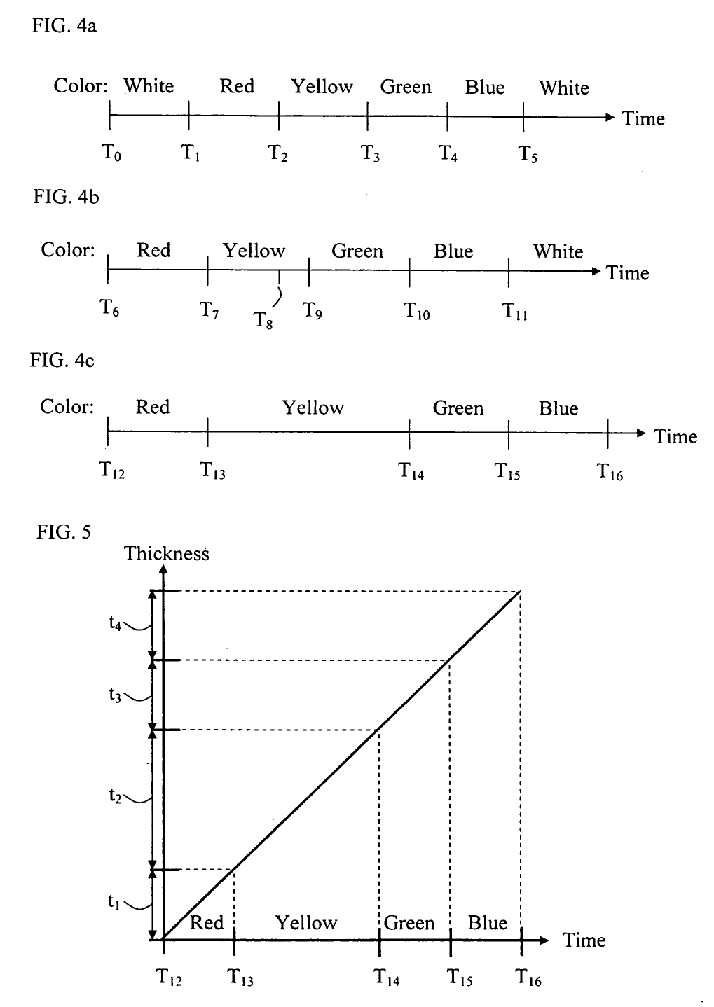

FIGS. 3a through 3f are simplified perspective views of particle 10 of FIGS. 2a and 2b as it changes color in response to the trigger and FIGS. 4a through 4c are simplified graphs showing how particle 10 changes color as a function of time. In FIG. 3a, particle 10 includes encapsulant 11 with regions 12-15 included therein as discussed above. In this example, encapsulant 11 is white so that particle 10 is white when the person starts brushing his or her teeth at a time T₀ until it dissolves at a time T₁ (See FIG. 4a). Here, encapsulant 11 and material regions 12-15 dissolve in response to moisture 20 (See FIG. 3b), which can be saliva, water, or combinations thereof. Near time T₁, encapsulant 11 is sufficiently enough dissolved to expose material region 12 and red particles 22 so that particle 10 appears red between times T₁ and T₂.

It should be noted here and in the following discussion that the change in color can happen gradually with time as one material region dissolves to reveal another material region. Hence, during some intervals of time the color of particles 10 can be a combination of one or more colors wherein one color dissipates in intensity as its region dissolves and another color increases in intensity as its region is revealed. For example, near time T₁, the color of particle 10 can be a combination of white and red where the white color tends to dominate before time T₁ and the red color tends to dominate after T₁.

Near time T₂, material region 12 is dissolved to expose material region 13 and yellow particles 23 so that particle 10 appears yellow between times T₂ and T₃. Near time T₃, material region 13 is dissolved to expose material region 14 and green particles 24 so that particle 10 appears green between times T₃ and T₄. Near time T₄, material region 14 is dissolved to expose material region 15 and blue particles 25 so that particle 10 appears blue between times T₄ and T₅. Near time T₅, material region 15 dissolves and breaks apart into particles 26. In this particular example, particles 26 blend into carrier material 32 so that dentrifrice has the color of carrier material 32, which is white here, after time T₅. In other examples, particles 26 can reflect a color of light, such as orange, which is different from the colors of regions 11-15.

FIG. 4b shows that there can be a waiting period while portions of particle 10 dissolve. In this example, encapsulant 11, if included, has dissolved near a time T₆ to expose material region 12 and red particles 22. Near time T₇, material region 12 is dissolved to expose material region 13 and yellow particles 23 so that particle 10 appears yellow between times T₇ and T₉. During this time period, however, the person can wait between a time T₈ and T₉ until material region 13 dissolves. Near time T₉, material region 13 is dissolved to expose material region 14 and green particles 24 so that particle 10 appears green between times T₉ and T₁₀. Near time T₁₀, material region 14 is dissolved to expose material region 15 and blue particles 25 so that particle 10 appears blue between times T₁₀ and T₁₁. Near time T₁₁, material region 15 dissolves and breaks apart into particles 26. In this particular example, particles 26 blend into carrier material 32, as discussed above. Here, dentrifrice has the color of carrier material 32, which is white, after time T₁₁.

It should be noted that the order of the color of particle 10 can be different in other examples. Hence, the sequence of particle 10 being red, yellow, green, and blue as a function of time is for illustrative purposes. It should also be noted that the amount of time that a particular color appears can also be different for different colors and will generally depend on the thickness of the corresponding colored material region. This is better illustrated in FIG. 4c where material region 13 is thicker than regions 11-12 and 14-15 so that particle 10 is yellow for a longer amount of time. In this example, encapsulant 11, if included, has dissolved near a time T₁₂ to expose material region 12 and red particles 22. Near time T₁₃, material region 12 is dissolved to expose material region 13 and yellow particles 23 so that particle 10 appears yellow between times T₁₃ and T₁₄. This time period, however, is longer than the time period between times T₁₂ and T₁₃ because thickness t₂ is greater than thickness t₁.

Near time T₁₄, material region 13 is dissolved to expose material region 14 and green particles 24 so that particle 10 appears green between times T₁₄ and T₁₅. The time period between times T₁₃ and T₁₄ is also longer than the time period between times T₁₄ and T₁₅ because thickness t₂ is greater than thickness t₃ (See FIG. 2a). Near time T₁₅, material region 14 is dissolved to expose material region 15 and blue particles 25 so that particle 10 appears blue between times T₁₅ and T₁₆. The time period between times T₁₃ and T₁₄ is longer than the time period between times T₁₅ and T₁₆ because thickness t₂ is greater than thickness t₄. Near time T₁₆, material region 15 dissolves and breaks apart into particles 26. In this particular example, particles 26 blend into carrier material 32, as discussed above. Hence, dentrifrice 10 is white after time T₁₆ so that it has the color of carrier material 32.

FIG. 5 is a simplified graph showing how the thicknesses of material regions 12-15 is related to the color change time. Here, the time period between times T₁₃ and T₁₄ is greater than each of the time periods between times T₁₅ and T₁₆, times T₁₄ and T₁₅, and times T₁₅ and T₁₆, as discussed above. In this way, thickness t₂ is greater than each of thicknesses t₁, t₃, and t₄ so that particle 10 is yellow for a longer period of time and red, green, and yellow for shorter periods of time. In one example, the period of time between times T₁₂ and T₁₃, times T₁₄ and T₁₅, and times T₁₅ and T₁₆ is each about 30 seconds and the period of time between times T₁₃ and T₁₄ is about 45 seconds, so that the total brushing time is indicated to be about 2 minutes and 15 seconds.

As mentioned above and as shown in FIG. 2b, particle 10 can be spherical or it can have a different shape in other examples. One such example is illustrated in FIGS. 6a and 6b which shows a particle 90. FIG. 6a is a simplified perspective view of an embodiment of particle 90 in accordance with the present invention and FIG. 6b is a simplified partial cut-away view of particle 90. In this embodiment, d₁ is greater than d₂ (See FIG. 6b) so that particle 90 is pill shaped. Particle 90 includes an outer encapsulant 91 which can be the same or similar to encapsulant 11 of FIG. 2a. Encapsulant 91 surrounds a multiple color material region 92, as shown in FIG. 6b. Region 92 includes a region 93 of red material which surrounds a region 94 of blue material. Region 92 also includes a region 95 of green material which is encapsulated by region 94. In this particular example, region 93 includes a central body portion 97 with a plurality of arms extending therefrom and regions 94 and 95 are diamond shaped. It should be noted, however, that regions 93, 94, and 95 can have other shapes.

In this embodiment, particle 90 dissolves in response to a trigger so that it changes color in much the same way as particle 10 discussed above. Here, the trigger is moisture 96 which comes into contact with outer encapsulant 91 so that it starts to dissolve. After a first period of time, substantially all of encapsulant 91 is dissolved away to expose multiple color material region 92 so that region 93 is visible and particle 90 appears red. After a second period of time, substantially all of region 93 of red material is dissolved away to expose region 94 of blue material so that particle 90 appears blue. After a third period of time, substantially all of region 94 is dissolved away to expose region 95 of green material so that particle 90 appears green. It should be noted that during the first, second, and third periods of time, particle 90 appears substantially red, blue, and green, respectively, although during certain intervals, particle 90 can appear to be a mixture of more than one color. In this way, the time that the person has brushed his or her teeth can be determined as discussed above in conjunction with FIGS. 4a-4c and FIG. 5.

It should be noted that in some embodiments, dentrifrice 30 and/or particle 90 can include other materials to aid in the care of ones teeth. For example, the material can include a medicament, fluoride, a plaque fighting substance, or a teeth whitening agent, among others. In any of these examples, the material can be released as a function of time as particle 10 or 90 changes color. For dentrifrice 30, the material can be included in the mixture of carrier material 32 and/or particles 10. In one example of dentrifrice 30, outer encapsulant 11 includes a visible plaque indicator material so that the person brushing his or her teeth can see any plaque thereon. Blue core material region 15 can include a plaque neutralizer material to neutralize the visible plaque indicator material after the person is done brushing. For particle 90, the material can be in outer encapsulant 91, any one of regions 92 through 95, or combinations thereof. In one example of particle 90, outer encapsulant 91 includes a visible plaque indicator material so that the person brushing his or her teeth can see the plaque thereon. Region of green material 95 can include a plaque neutralizer material to neutralize the visible plaque indicator material after the person is done brushing. It should be noted that there are other examples of including a medicament, fluoride, a plaque fighting substance, or a teeth whitening agent in dentrifrice 30 and/or particle 90, but only a few are discussed here for ease of discussion.

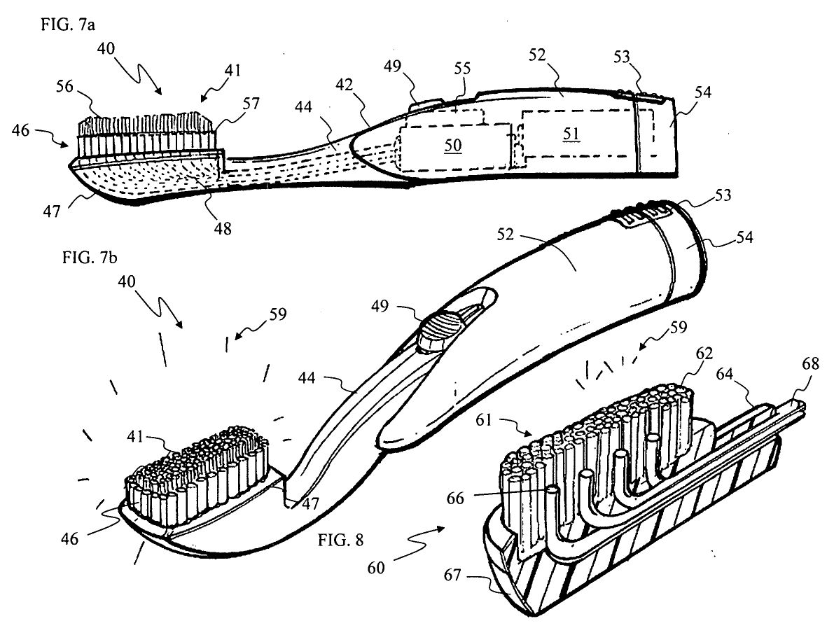

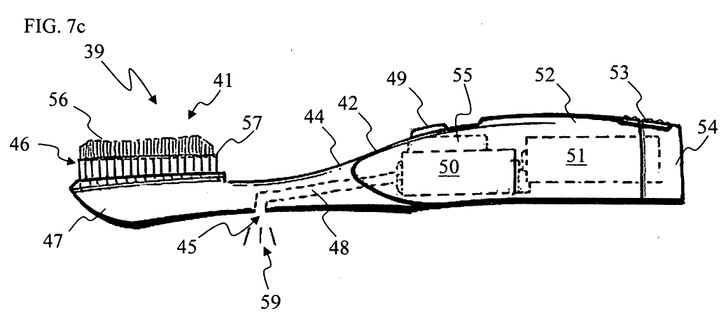

FIGS. 7a and 7b are simplified side and perspective views, respectively, of one embodiment of a tooth brush 40 which provides light 59. Toothbrush 40 includes an elongated body portion 42 having a head 47, a handle 52, and a neck 44 therebetween. Head 47 of toothbrush 40 includes toothbrush bristles 41 attached or embedded into head 47 in a manner well known in the art. A light source 50, control circuitry 55, and a power source 51 are positioned in handle 52. Light source 50 is electrically coupled to power source 51 and control circuitry 55. A switch 49 is positioned on an outer surface of handle 52 and coupled to control circuitry 55. Elongated body portion 43 includes an end cap 54 which allows access to power source 51. In this example, end cap 54 is held to handle 52 by a clasp 53. Light source 50 can be, for example, a light emitting diode (LED) or laser diode (LD). Power source 51 can include a battery or other power supply known in the art. The rated power of power source 51 should be sufficient to operate on light source 50 and control circuitry 55.

Light guiding elements 48 extend from light source 50 through neck 44 and into head 47. Elements 48 then extend upwardly through head 47 proximate to and along an outer periphery of bristles 41 (See FIG. 7b). Ends 57 of elements 48 extend below ends 56 of bristles 41 and emit light therethrough so that bristles 41 luminesce. Light guiding elements 48 can be, for example optical fibers or waveguides which guide light from light source 50 to bristles 41. The luminescent light is emitted from bristles 41 as light 59 (See FIG. 7b). The light emitted by ends 57 of elements 48 enhances the color of dentrifrice 30 on bristles 41 (not shown). In this way, the color change of particles 10 can be better seen by the person when brushing his or her teeth.

FIG. 7c is a simplified side view of another embodiment of a tooth brush 39 which provides light 59. Toothbrush 39 is similar to toothbrush 40 illustrated in FIGS. 7a and 7b. However, in this example, light guiding element 48 extends from light source 50 through neck 44 and downwardly to an opening 45 on a surface of neck 44 opposite to bristles 56. In this way, light source 50 emits light 59 through element 48 so that it is outputted from opening 45. Further, light source 50 emits light 59 so that its color changes as a function of time as the teeth are brushed. In one example, the light from opening 45 can be red for about 45 seconds, yellow for about 45 seconds, and blue for about 45 seconds, so that the total brushing time is about 2 minutes and 15 seconds. Light source 50 can then switch off or emit another color of light, such as white, to indicate that the person can stop brushing. Hence, the person brushing his or her teeth can tell the amount of time that has elapsed by the color of light 59 emitted from opening 45. Light source 50 can include different colored light bulbs or light emitting diodes to emit the appropriate color of light.

FIG. 8 is a simplified perspective view of another embodiment of a tooth brush 60 in accordance with the present invention. Tooth brush 60 includes a head 67 which extends from a neck 64. Bristles 61 are attached or embedded into head 67 in a manner well known in the art so that bristles 61 extend upwardly therefrom head 67. Light guiding elements 68 extend from a light emitter (not shown) through neck 64 and into head 67. Elements 68 can be the same or similar to elements 48 discussed above. Elements 68 then extend upwardly through head 67 proximate to and within an inner periphery of bristles 61. Ends 66 of optical fibers 68 extend below ends 62 of bristles 61 and emit light therethrough so that bristles 61 luminesce. The luminescent light is emitted from bristles 61 as light 59. The light emitted by ends 66 of optical fibers 68 enhances the color of dentrifrice 30 on bristles 61 (not shown). In this way, the color change of particles 10 can be better seen by the person when brushing his or her teeth.

FIG. 9 is a simplified perspective view of another embodiment of a tooth brush 70 in accordance with the present invention. Tooth brush 70 includes an elongated neck 74 with an optically clear head 77 coupled to it. Bristles 71 extend upwardly from head 77 and are attached or embedded thereto in a manner well known in the art. Light guiding elements 78 extend through elongated neck 74 and into head 77. Elements 78 have ends 75 which terminate in head 77 so that light 79 is emitted from ends 75 through head 77. Some of the light from ends 75 is directed upwardly through head 77 and bristles 71 so that bristles 71 luminesce. The luminescent light is emitted from bristles 71 as light 76. The light from ends 75 directed through bristles 71 enhances the color of dentrifrice 10 on bristles 71 (not shown). In this way, the color change of particles 30 can be better seen by the person when brushing his or her teeth. Further, light 79 enhances the appearance of optically clear head 77. In this example, bristles 71 can rotate in a direction 82 relative to head 77, but in other examples, bristles 71 can be stationary.

The present invention is described above with reference to a preferred embodiment. However, those skilled in the art will recognize that changes and modifications may be made in the described embodiment without departing from the nature and scope of the present invention. Various changes and modifications to the embodiment herein chosen for purposes of illustration will readily occur to those skilled in the art. To the extent that such modifications and variations do not depart from the spirit of the invention, they are intended to be included within the scope thereof.

Having fully described the invention in such clear and concise terms as to enable those skilled in the art to understand and practice the same, the invention claimed is:

1. Dentifrice, comprising:
a carrier material;
a plurality of particles in the carrier material, the plurality of particles including multiple regions of different colored materials; and
an outer region which at least partially encapsulates the multiple regions of different colored materials.

2. The dentifrice of claim 1, wherein the multiple regions of different colored materials are each spheroid in shape, the spheroid shapes being substantially concentric.

3. The dentifrice of claim 1, wherein the multiple regions of different colored materials change color in response to a trigger.

4. The dentifrice of claim 3, wherein the trigger includes at least one of moisture and friction.

5. The dentifrice of claim 1, wherein a thickness of each region in the multiple regions of different colored materials is chosen so that the region dissolves after a predetermined amount of time in response to a trigger.

6. The dentifrice of claim 1, further including at least one of a medicament, fluoride, a plaque indicator material, a plaque neutralizer material, and a teeth whitener.

7. A method of cleaning teeth, comprising:
providing a tooth brush which includes a plurality of bristles;
positioning a dentrifrice on the bristles of the tooth brush, the dentifrice including
a carrier material;
a plurality of particles in the carrier material, the plurality of particles including multiple regions of different colored materials; and
applying the dentrifrice to the teeth with the plurality of bristles, the particles in the plurality of particles changing color as they dissolve.

8. The method of claim 7, wherein the change in color of the plurality of particles indicates a time that the teeth have been brushed.

9. The method of claim 7, wherein a thickness of the regions in the multiple regions of dissolvable material is chosen so that the corresponding region dissolves in a predetermined amount of time.

10. The method of claim 7, wherein the multiple regions of different colored materials are each spheroid in shape.

11. The method of claim 7, wherein the tooth brush includes a light source which emits light.

12. The method of claim 7, further including a step of waiting until the color of the particles in the plurality of particles changes.

13. The dentifrice of claim 7, wherein the multiple regions of different colored materials are dissolvable in response to a trigger.

14. A method of making a dentrifrice, comprising:
providing a carrier material;
providing a plurality of particles in the carrier material, the plurality of particles including multiple regions of different colored materials; and
mixing the plurality of particles and the carrier material together.

15. The method of claim 14, wherein the step of providing the plurality of particles includes encapsulating a first material region in the multiple regions of different colored materials with a second material region, the first and second material regions having different colors.

16. The method of claim 14, wherein the multiple regions of different colored materials are spheroid in shape.

17. The method of claim 14, further including providing a light reflecting material in the multiple regions of different colored materials.

18. The method of claim 14, further including providing at least one of a medicament, fluoride, and a teeth whitening agent in the mixture of the carrier material and the plurality of particles.

19. The method of claim 14, further including providing an outer encapsulating region around the multiple regions of different colored materials.

20. The method of claim 14, further including choosing a thickness of each region in the multiple regions of different colored materials so that the corresponding region dissolves in a predetermined amount of time.
